(19) United States
(12) Patent Application Publication
(10) Pub. No.: US 2026/0028293 A1
(43) Pub. Date: Jan. 29, 2026

(54) TREATMENT OF ORGANIC WASTE

(71) Applicant: BIOMAX HOLDINGS PTE LTD, Singapore (SG)

(72) Inventors: Eng Tong SIM, Singapore (SG); Chum Mok PUAH, Singapore (SG)

(21) Appl. No.: 18/996,494

(22) PCT Filed: Jul. 24, 2023
(86) PCT No.: PCT/SG2023/050511

§ 371 Date: Jan. 17, 2025

(30) Foreign Application Priority Data
Aug. 19, 2022 (SG) 10202250785G

(51) Int. Cl. C05F 17/20; C05F 11/08; C05F 17/60; C12N 1/20; C12R 1/445; C12R 1/45

(52) U.S. Cl. CPC C05F 17/20; C05F 11/08; C05F 17/60; C12N 1/205; C12R 2001/445; C12R 2001/45

(57) ABSTRACT

The present invention relates to a method of treating organic waste comprising the steps of: treating the organic waste with a microorganism composition comprising the following microorganism species: Niallia sp., Stenotrophomonas sp. and Staphylococcus sp., wherein the treatment step is conducted under aerobic environment with stirring to convert the organic waste to an organic fertilizer.

### FIELD OF INVENTION

The present invention relates to waste management. Particularly, the present invention relates to treatment of organic waste and more particularly, rapid thermophilic treatment of organic waste. The present invention also relates to an organic fertilizer derived from the treatment of organic waste.

### BACKGROUND OF THE INVENTION

Organic waste such as food waste and agricultural waste is generated in large quantities and an increasing trend in the amount of waste generation is observed. Waste management is now becoming a huge problem. Most of the organic waste is either burnt or disposed in landfills or ocean. Organic waste in landfills undergoes biodegradation over time and tremendous amount of greenhouse gases such as methane are generated, thereby contributing to increase in climate temperature (greenhouse effect). Harmful materials such as antibiotics, chemicals and pathogens may leach from the organic waste and enter soil and groundwater as the organic waste breaks down.

It is crucial to find ways for proper management of the organic waste. There have been attempts to convert or recycle organic waste into useful goods or products, although this merely constitutes a small proportion of the organic waste generated. Activities involving recycling of the organic waste are gradually increasing due to recent increase in public awareness on waste management, however, this is still unsatisfactory due to various limitations on the available ways of recycling organic waste.

One example of recycling organic waste is to convert it into energy via digestion, where waste is subjected to an oxygen-depleted environment while it breaks down, to create an alternative energy source called biogas. Sludge is generated as by-product of the biogas production and it can be used to fertilize and improve soil, and inactivate pathogens in the organic waste. Nevertheless, biogas production usually requires high capital cost and long processing time, and not all types of organic waste can be treated this way. Other alternative uses of organic waste include fish farming, production of algae for human and animal consumption and production of aquatic macrophytes for animal feed supplements. Organic waste is also often undergone composting until it becomes nutrient-rich fertilizers or soil conditioners and soil amendments.

Composting converts organic waste into harmless and value-added products by a microbiological process. Composting is usually done naturally by using naturally-occurring microorganisms and typically no harmful by-products are produced and therefore post-process treatment is usually not required. However, natural composting can easily take up to months and even years to mature, and typically results in a product with relatively low nitrogen, phosphorus and potassium (NPK) content (typically less than 1:1:1). Organic fertilizers with low NPK content are less useful and less valuable.

At present, various biological treatments are available to increase the speed of composting. One of these treatments is disclosed in Chinese patent no. CN 112760274 B. Liquid or solid preparation containing Puntoea sp. is used in aerobic composting of solid organic waste which takes about 10 days or longer to mature. Whereas Japanese patent no. JP 5445821 B2 discloses the use of microbial composition containing microorganisms selected from Paenibacillus sp., Bacillus sp. and Brevibacterium sp. in decomposition of organic waste with a relatively shorter treatment period, i.e. 3 to 5 days. In PCT publication no. WO 2011/119112 A1, it discloses a process for treatment of organic waste comprising the step of contacting the organic waste with at least three of Bacillus sp. microorganisms, Pseudomonas sp. microorganisms, Bifidobacterium sp. microorganisms and Lactobacillus sp. Microorganisms under conditions to at least partly convert the organic waste to organic fertilizer. Yet, this process still requires at least 24 hours to mature.

Although the available biological treatments take a relatively shorter period of time to mature as compared to natural composting, its efficiency is still unsatisfactory considering the amount of organic waste produced is tremendous. Accordingly, there is a need to provide an efficient, simple, cost-effective and environmentally friendly process for treatment of organic waste.

### SUMMARY OF INVENTION

According to one aspect of the present invention, there is provided a method of treating organic waste comprising the steps of: treating the organic waste with a microorganism composition comprising the following microorganism species: Niallia sp., Stenotrophomonas sp. and Staphylococcus sp., wherein the treatment step is conducted under aerobic environment with stirring to convert the organic waste to an organic fertilizer

Preferably, an initial moisture content of the organic waste is controlled at 25 wt % to 70 wt %, preferably at 30 wt % to 65 wt % and more preferably about 45 wt %.

Preferably, the Niallia sp. is Niallia circulans, Niallia endozanthoxylica, Niallia nealsonii, Niallia taxi and Niallia oryzisdi, the Stenotrophomonas sp. is Stenotrophomonas maltophilia, Stenotrophomonas rhizophilia, Stenotrophomonas africana and Stenotrophomonas acidaminiphlia, and the Staphylo coccus sp. is Staphylococcus haemolyticus, Staphylococcus aureus, Staphylococcus intermedius, Staphylococcus epidermidis and Staphylococcus simulans.

Preferably, a weight ratio of the microorganism composition to organic waste is 1:1000 to 2:1000.

Preferably, the treatment step is conducted at a temperature of 60° C. to 85° C.

Preferably, the treatment step is conducted with intermittent aeration, and more preferably, each cycle of aeration is conducted for 5 minutes to 45 minutes and suspended for 5 minutes to 45 minutes.

Preferably, a rotational speed of the stirring is 3 rpm to 10 rpm.

Preferably, prior to the treatment step, the method further comprises a step of reducing size of the organic waste.

Preferably, the microorganism composition is a powder comprising microorganisms in an amount of 1×10¹⁰ cfu per gram of powder to 15×10¹⁰ cfu per gram of powder. More preferably, the microorganism composition comprises a bulking agent selected from rice bran, ash, water, skim milk powder, corn grits or any combinations thereof.

Alternatively, the microorganism composition is a solution comprising 5 vol % to 50 vol % of microorganisms.

Preferably, the treatment step is carried out for 12 hours or less.

The preferred embodiment of the invention consists of novel features and a combination of parts hereinafter fully described and illustrated in the accompanying drawings and particularly pointed out in the appended claims; it being understood that various changes in the details may be effected by those skilled in the arts but without departing from the scope of the invention or sacrificing any of the advantages of the present invention.

### DETAILED DESCRIPTION OF THE INVENTION

Hereinafter, the invention shall be described according to the preferred embodiments of the present invention and by referring to the accompanying description and drawings. However, it is to be understood that limiting the description to the preferred embodiments of the invention and to the drawings is merely to facilitate discussion of the present invention and it is envisioned that those skilled in the art may devise various modifications without departing from the scope of the appended claims.

The present invention relates to waste management. Particularly, the present invention relates to treatment of organic waste and more particularly, rapid thermophilic treatment of organic waste. The present invention also relates to an organic fertilizer derived from the treatment of organic waste.

### Organic Waste

The term “organic waste” as used herein refers to any biodegradable carbon-containing material and comes from either a plant or an animal. Organic waste includes, but is not limited to, agricultural waste, food waste, organic refuse, mill effluent, municipal waste, sewage, sludge, animal waste, industrial waste or any combination thereof. Example of agricultural waste includes, but is not limited to, empty fruit bunch (EFB), palm decanter cake slurry, olive husk, corn cob, coffee bean husk, rice husk, rice straw, spent mushroom compost, palm foliage, palm trunk, palm kernel shells, palm fibre, farm effluent, slaughterhouse waste, flower cuttings, spent flower compost, wheat straw, fruit waste, vegetable waste and the like. Example of animal waste includes, but is not limited to, dead animals, animal feather, animal parts (such as animal intestines) and animal manure such as poultry manure, cow manure, goat manure, horse manure, sheep manure and swine manure.

One or more types of organic waste may be used. For example, EFB may be used together with chicken manure, or food waste may be used together with rice husk. Other exemplary combination of organic wastes includes, but is not limited to, a combination of chicken manure with saw dust, a combination of chicken manure with chicken feathers, a combination of EFB with chicken manure, and a combination of EFB and palm oil mill effluent (POME).

### Microorganism Composition

The microorganisms useful in the disclosed process are those that are capable of degrading carbon compounds or fixing nitrogen compounds. Advantageously, mixed cultures of the microorganisms are used in order to obtain a broad spectrum of degradation or fixation.

In one embodiment, there is provided a composition comprising at least one microorganism from each of the following microorganism species: Niallia sp., Stenotrophomonas sp. and Staphylococcus sp.

The Niallia sp. microorganism is preferably selected from the group consisting of Niallia circulans, Niallia endozanthoxylica, Niallia nealsonii, Niallia taxi and Niallia oryzisdi. The Stenotrophomonas sp. microorganism is preferably selected from the group consisting of Stenotrophomonas maltophilia, Stenotrophomonas rhizophilia, Stenotrophomonas africana and Stenotrophomonas acidaminiphlia. The Staphylococcus sp. microorganism is preferably selected from the group consisting of Staphylococcus haemolyticus, Staphylococcus aureus, Staphylococcus intermedius, Staphylococcus epidermidis and Staphylococcus simulans.

Each of the Niallia sp., Stenotrophomonas sp. and Staphylococcus sp. may comprise one, two, three, four or more microorganisms from the same species.

The selection of the microorganisms may be dependent on the type of organic waste that is to be treated.

The microorganism composition may be in solution form although powder form is preferred for better stability of the microorganisms. The microorganism solution is preferably kept cool at a temperature of 0° C. to 10° C. for long term storage to ensure there is sufficient amount of microorganisms in their viable state. Whereas, the microorganism powder can be stored at room temperature provided it is away from sunlight and rain.

The microorganism content of the microorganism solution may comprise about 5 vol % to about 50 vol % microorganisms, about 10 vol % to about 50 vol % microorganisms, about 15 vol % to about 50 vol % microorganisms, about 20 vol % to about 50 vol % microorganisms, about 25 vol % to about 50 vol % microorganisms, about 30 vol % to about 50 vol % microorganisms, about 35 vol % to about 50 vol % microorganisms, about 40 vol % to about 50 vol % microorganisms, about 45 vol % to about 50 vol % microorganisms, about 5 vol % to about 40 vol % microorganisms, about 5 vol % to about 30 vol % microorganisms, about 5 vol % to about 20 vol % microorganisms, or about 5 vol % to about 15 vol % microorganisms in a microorganism culture. In a preferred embodiment, the microorganism content of the microorganism solution comprises about 10 vol % to about 20 vol %.

Additive for enhancing the efficacy and efficiency of the waste treatment (such as biological catalyst, buffers, nutrients and surfactants) is generally not required to be added to the microorganism composition.

The microorganism powder comprises a blend of microorganisms with a bulking agent or a carrier. The bulking agent or carrier includes, but is not limited to, ash, lentil powder, soybean powder, sugar cane pulp, fish meal powder, corn grits, rice bran, wheat bran, skim milk powder, charcoal powder or any combination thereof. Other suitable bulking agent can be used so long the bulking agent is able to create a powdery mixture with the microorganisms, and at the same time, it does not affect the viability of the microorganisms. Preferably, the bulking agent is from natural origin and does not release toxic gases or compounds. One or more types of bulking agent can be used as the bulking agent or carrier. For example, the bulking agent may comprises one, two, three, four, five, six or more types of bulking agent.

The microorganism content of the microorganism powder may comprise microorganism in an amount of from about 1×10¹⁰ cfu per gram of powder to about 15×10¹⁰ cfu per gram of powder, from about 1×10¹⁰ cfu per gram of powder to about 14×10¹⁰ cfu per gram of powder, from about 1×10¹⁰ cfu per gram of powder to about 13×10¹⁰ cfu per gram of powder, from about 1×10¹⁰ cfu per gram of powder to about 12×10¹⁰ cfu per gram of powder, from about 1×10¹⁰ cfu per gram of powder to about 11×10¹⁰ cfu per gram of powder, from about 1×10¹⁰ cfu per gram of powder to about 10×10¹⁰ cfu per gram of powder, from about 1×10¹⁰ cfu per gram of powder to about 9×10¹⁰ cfu per gram of powder, from about 1×10¹⁰ cfu per gram of powder to about 8×10¹⁰ cfu per gram of powder, from about 1×10¹⁰ cfu per gram of powder to about 7×10¹⁰ cfu per gram of powder, from about 1×10¹⁰ cfu per gram of powder to about 6×10¹⁰ cfu per gram of powder, from about 1×10¹⁰ cfu per gram of powder to about 5×10¹⁰ cfu per gram of powder, from about 1×10¹⁰ cfu per gram of powder to about 4×10¹⁰ cfu per gram of powder, from about 1×10¹⁰ cfu per gram of powder to about 3×10¹⁰ cfu per gram of powder, from about 1×10¹⁰ cfu per gram of powder to about 2×10¹⁰ cfu per gram of powder, from about 2×10¹⁰ cfu per gram of powder to about 15×10¹⁰ cfu per gram of powder, from about 3×10¹⁰ cfu per gram of powder to about 15×10¹⁰ cfu per gram of powder, from about 4×10¹⁰ cfu per gram of powder to about 15×10¹⁰ cfu per gram of powder, from about 5×10¹⁰ cfu per gram of powder to about 15×10¹⁰ cfu per gram of powder, from about 6×10¹⁰ cfu per gram of powder to about 15×10¹⁰ cfu per gram of powder, from about 7×10¹⁰ cfu per gram of powder to about 15×10¹⁰ cfu per gram of powder, from about 8×10¹⁰ cfu per gram of powder to about 15×10¹⁰ cfu per gram of powder, from about 9×10¹⁰ cfu per gram of powder to about 15×10¹⁰ cfu per gram of powder, from about 10×10¹⁰ cfu per gram of powder to about 15×10¹⁰ cfu per gram of powder, from about 11×10¹⁰ cfu per gram of powder to about 15×10¹⁰ cfu per gram of powder, from about 12×10¹⁰ cfu per gram of powder to about 15×10¹⁰ cfu per gram of powder, from about 13×10¹⁰ cfu per gram of powder to about 15×10¹⁰ cfu per gram of powder, or from about 14×10¹⁰ cfu per gram of powder to about 15×10¹⁰ cfu per gram of powder.

### Pre-Treatment of Organic Waste

Prior to treatment, pre-processing of the organic waste may be required. For examples, the particle size of solid organic waste may be reduced to facilitate the treatment process. Typically, the particle size of the organic waste can be reduced to about 1 mm to about 20 mm, and preferably, from about 5 mm to about 10 mm.

The moisture content of organic waste varies widely depending on the source, and it determines the availability of waste material that can be potentially converted to organic fertilizer. In this regard, the initial moisture content of the organic waste may be controlled within or adjusted to, for example, about 25 wt % to about 70 wt %, about 25 wt % to about 60 wt %, about 25 wt % to about 50 wt %, about 25 wt % to about 40 wt %, about 25 wt % to about 35 wt %, about 25 wt % to about 30 wt %, about 30 wt % to about 70 wt %, about 40 wt % to about 70 wt %, about 50 wt % to about 70 wt %, about 60 wt % to about 70 wt %, about 65 wt % to about 70 wt %, about 30 wt % to about 65 wt %, about 35 wt % to about 60 wt %, about 40 wt % to about 55 wt %, or about 45 wt % to about 50 wt %. Preferably, the initial moisture content of the organic waste is about 30 wt % to about 65 wt % and more preferably about 45 wt %. The initial moisture content of the organic waste may be controlled or adjusted by any means that are well known to those skilled in the art.

The organic waste may optionally be combined with other additives or nutrients to enhance the treatment process and/or to increase the nutrient level of the organic fertilizer produced by the treatment process. Such additives may be, for example, carbon sources such as ash, saw dust, dried leaves, wood shavings, rice stalks, waste papers and the like.

pH control is generally not required and the treatment process can be conducted at the pH value of the organic waste used.

### Treatment of Organic Waste

The organic waste is treated with the microorganism composition by seeding the organic waste with the microorganism composition, wherein the treatment is conducted under aerobic environment with stirring to convert the organic waste to an organic fertilizer. Gentle stirring at about 3 rpm to 10 rpm (e.g. 5 rpm) is typically applied. Monitoring or controlling of the moisture content of the organic waste is not necessary once the treatment has started, i.e. upon contact of the organic waste with the microorganism composition.

Contact of the organic waste with the microorganism composition results in degradation or conversion of chemical compounds contained within the organic waste so as to neutralize odorous compounds contained therein and render the organic waste odorless, or conversion of the carbon-compounds or nitrogen fixation to increase the nutrient level of the organic waste.

Preferably, the weight ratio of the microorganism composition to organic waste is at least 1:1000. A higher weight ratio of microorganism composition to organic waste may be applied to improve the efficiency of the treatment process, for example, 1.1:1000, 1.2:1000, 1.3:1000, 1.4:1000, 1.5:1000, 1.6:1000, 1.7:1000, 1.8:1000, 1.9:1000, 2:1000, 2.5:1000, 3:1000, 3.5:1000, 4:1000, 4.5:1000 or 5:1000.

The treatment is carried out at a temperature of, for example, about 60° C. to about 85° C., about 65° C. to about 85° C., about 70° C. to about 85° C., about 75° C. to about 85° C., about 80° C. to about 85° C. Unwanted microorganisms such as Shigella sp. microorganisms, Salmonella sp. and E. coli will be removed or destroyed during the treatment process at this temperature range. Usually, the treatment process will generate certain extent of heat, typically at temperature of about 73° C., hence, temperature monitoring may not be necessary after this.

The treatment period required may depend on factors such as the initial NPK level and/or C:N ratio of the organic waste to be treated, the type and concentration of the microorganisms used, and the treatment conditions applied in the process. Typically, the organic waste is treated with the microorganism composition for a period of time of about 12 hours or less. For example, the treatment period may be about 5 hours to about 12 hours, about 6 hours to about 12 hours, about 7 hours to about 12 hours, about 8 hours to about 12 hours, about 9 hours to about 12 hours, about 10 hours to about 12 hours, about 11 hours to about 12 hours, about 5 hours to about 11 hours, about 5 hours to about 10 hours, about 5 hours to about 9 hours, about 5 hours to about 8 hours, about 5 hours to about 7 hours, or about 5 hours to about 6 hours. As the treatment period only lasts 12 hours or less, dosing is usually not required.

Advantageously, in about 12 hours, an NPK content of about 1-5:1-5:1-7 and a C:N ratio of about 5:1 to about 30:1 may be achieved. In general, a higher NPK content results in a more effective fertilizer in that a lesser amount of fertilizer is required to be used to promote plant growth compared to a fertilizer having a lower NPK content. An organic fertilizer with a higher NPK content is therefore more cost-effective than an organic fertilizer with a lower NPK content. Whereas a lower C:N ratio is preferred for use as fertilizer to promote plant growth.

Preferably, the treatment is conducted with aeration. Aeration may be provided continuously throughout the treatment, or intermittently/periodically. Air may be pumped in for about 5 minutes to 45 minutes, for example, for 5 minutes, 10 minutes, 15 minutes, 20 minutes, 25 minutes, 30 minutes, 35 minutes, 40 minutes or 45 minutes, suspended for about to 5 minutes to 45 minutes, for example, for 5 minutes, 10 minutes, 15 minutes, 20 minutes, 25 minutes, 30 minutes, 35 minutes, 40 minutes or 45 minutes, and pumped in again for about 5 minutes, 10 minutes, 15 minutes, 20 minutes, 25 minutes, 30 minutes, 35 minutes, 40 minutes or 45 minutes.

After treatment of the organic waste, the treated organic waste product is left to cool at room temperature typically for 1 day to about 2 days, to yield an organic fertilizer product prior to being packaged.

Example of apparatus that can be used for carrying out the present treatment process is as disclosed in PCT publication no. WO 2011/119112 A1.

### EXAMPLE

### (A) Preparation of Microorganism Composition

1 L of nutrient broth was prepared by mixing 10 g glucose, 8 g yeast extract and 5 g sodium chloride. The nutrient broth was then inoculated with the selected microorganisms. The microorganism compositions in Table 1 are prepared. The inoculated nutrient broths were cultured at 35° C.

TABLE 1

| Microorganism | | B |
|---|---|---|
| Composition | A | (Present Invention) |
| Microorganisms | Streptomyces pactum | Niallia circulans |
| | Corynebacterium | Stenotrophomonas |
| | striatum | maltophilia |
| | Bacillus pumilus | Staphylococcus |
| | | haemolyticus |

### (B) Analytical Methods

### NPK Content

Standard Kjeldahl's method (APHA 4500 N_{org} B) was used to determine the total nitrogen content in the organic fertilizer. Standard acid digestion of the organic fertilizer followed by inductively coupled plasma atomic emission spectroscopy (ICP-AES) was used to determine the phosphorus and potassium contents in the organic fertilizer.

### C:N Ratio

The organic carbon content was determined using the standard Loss on Ignition (LOI) method. A sample of the organic waste or organic fertilizer was weighed and its initial weight recorded. The sample was then placed in an oven at 350° C. for 3 hours. The sample was then cooled, re-weighed, and its final weight is recorded. The organic carbon content was determined as follows:

$$\% Organic Carbon = ( Loss in weight \div Initial Weight ) \times 100$$

The nitrogen content was determined using standard Kjeldahl's method (APHA 4500 N_{org} B).

The C:N ratio was then determined as follows:

$$C : N Ratio = \% Organic Carbon : \% Nitrogen$$

(C) Preparation of Organic Fertilizer from Raw Chicken Manure

Raw chicken manure was mixed with saw dust. The initial moisture content of the organic waste mixture was adjusted to about 45 wt %. The microorganism composition as prepared above was added to the mixture to obtain the samples in Table 2.

TABLE 2

| Sample | 1 | 2 | 3 |
|---|---|---|---|
| Microorganism | A | A | B |
| Composition | | | |
| Organic Waste | Raw chicken manure (80 wt %) | | |
| | Saw dust (20 wt %) | | |
| Treatment Time | 24 hours | 12 hours | 12 hours |

After mixing the mixture and microorganism, air was pumped in for 5 min, stopped for 40 min, and resumed for another 5 min to maintain an aerobic environment. The process was allowed to run for a period of time as stated in Table 2 and then cooled for 2 days. The NPK content and C:N ratios were determined using the analytical methods set out above. The analytical results of the treated samples are tabulated in Table 3.

TABLE 3

| Sample | 1 | 2 | 3 |
|---|---|---|---|
| N (wt %) | 1.5 | 1.4 | 1.1 |
| P (wt %) | 1.9 | 5.7 | 5.6 |
| K (wt %) | 1.6 | 2.0 | 1.2 |
| C:N ratio (before | 45 | 45 | 45 |
| treatment | | | |
| C:N ratio (after | 16 | 17 | 20 |
| treatment | | | |
| Odour | Odourless | Odour | Odourless |
| Texture | Good | Not Good | Good |

It could be seen in Table 3 that all treated samples contain reasonable high NPK content/content while the C:N ratio was significantly reduced. However, the odour and texture quality of sample 2 are unsatisfactory with 12 hours treatment time. A longer period of time is required (sample 1, 24 hours) to eliminate the foul odour of the organic waste and achieve the desired texture. On the other hand, sample 3 using the present microorganism composition is able to achieve high NPK content, reduce C:N ratio, eliminate foul odour and achieve the desired texture within 12 hours treatment period.

The present disclosure includes as contained in the appended claims, as well as that of the foregoing description. Although this invention has been described in its preferred form with a degree of particularity, it is understood that the present disclosure of the preferred form has been made only by way of example and that numerous changes in the details of construction and the combination and arrangements of parts may be resorted to without departing from the scope of the invention.

1. A method of treating organic waste comprising the steps of:
treating the organic waste with a microorganism composition comprising at least one microorganism from each of the following microorganism species: Niallia sp., Stenotrophomonas sp. and Staphylococcus sp.,
wherein the treatment step is conducted under aerobic environment with stirring to convert the organic waste to an organic fertilizer.

2. The method according to claim 1, wherein an initial moisture content of the organic waste is controlled at 25 wt % to 70 wt %.

3. The method according to claim 2, wherein the initial moisture content of the organic waste is controlled at 30 wt % to 65 wt %.

4. The method according to claim 1, wherein the Niallia sp. is Niallia circulans, Niallia endozanthoxylica, Niallia nealsonii, Niallia taxi and Niallia oryzisdi.

5. The method according to claim 1, wherein the Stenotrophomonas sp. is Stenotrophomonas maltophilia, Stenotrophomonas rhizophilia, Stenotrophomonas africana and Stenotrophomonas acidaminiphlia.

6. The method according to claim 1, wherein the Staphylococcus sp. is Staphylococcus haemolyticus, Staphylococcus aureus, Staphylococcus intermedius, Staphylococcus epidermidis and Staphylococcus simulans.

7. The method according to claim 1, wherein a weight ratio of the microorganism composition to organic waste is 1:1000.

8. The method according to claim 1, wherein the treatment step is conducted at a temperature of 60° C. to 85° C.

9. The method according to claim 1, wherein the treatment step is conducted with intermittent aeration.

10. The method according to claim 9, wherein each cycle of aeration is conducted for 5 minutes to 45 minutes and suspended for 5 minutes to 45 minutes.

11. The method according to claim 1, wherein a rotational speed of the stirring is 3 rpm to 10 rpm.

12. The method according to claim 1 further comprising, prior to the treatment step, a step of reducing size of the organic waste.

13. The method according to claim 1, wherein the microorganism composition is a powder.

14. The method according to claim 13, wherein the microorganism composition comprises microorganisms in an amount of 1×10¹⁰ cfu per gram of powder to 15×10¹⁰ cfu per gram of powder.

15. The method according to claim 13, wherein the microorganism composition further comprises a bulking agent.

16. The method according claim 15, wherein the bulking agent is selected from rice bran, ash, water, skim milk powder, corn grits or any combinations thereof.

17. The method according to claim 1, wherein the microorganism composition is a solution.

18. The method according to claim 17, wherein the microorganism composition comprises 5 vol % to 50 vol % of microorganisms.

19. The method according to claim 1, wherein the treatment step is carried out for 12 hours or less.
